(19) United States
(12) Patent Application Publication
(10) Pub. No.: US 2005/0195360 A1
(43) Pub. Date: Sep. 8, 2005

(54) Ophthalmic apparatus

(75) Inventors: Junichi Akita, Nukata-gun (JP); Naoyuki Kondoh, Anjo-shi (JP); Toshifumi Sumiya, Nukata-gun (JP); Susumu Oshima, Ama-gun (JP); Akihiro Fujishiro, Toyohashi-shi (JP); Toshiaki Mizuno, Gamagori-shi (JP)

Correspondence Address: OLIFF & BERRIDGE, PLC, P.O. BOX 19928, ALEXANDRIA, VA, 22320, US

(73) Assignee: NIDEK CO., LTD., Gamagori-shi (JP)

(21) Appl. No.: 11/066,139

(22) Filed: Feb. 25, 2005

(30) Foreign Application Priority Data
Mar. 1, 2004 (JP) 2004-055612

(51) Int. Cl. A61B 3/10

(52) U.S. Cl. 351/212; 351/221; 351/214

(57) ABSTRACT

An ophthalmic apparatus for examining an eye of a patient, comprises: a light projecting optical system for projecting examination light onto the eye, the optical system including: a light source; and a projection shape forming device which is electrically controlled to change a reflecting area of a reflecting surface or a transmitting area of a transmitting surface and is arranged to reflect or transmit the examination light emitted from the light source while forming a projection shape of the examination light into a desired shape so that the examination light of the desired shape is projected onto the eye; and a selection unit for selecting the projection shape of the examination light; and a control part which drivingly controls the forming device based on the selected projection shape.

### BACKGROUND OF THE INVENTION

1. Field of the Invention

The present invention relates to an ophthalmic apparatus for examining (including observing, measuring, and others) a patient's eye.

2. Description of Related Art

There are ophthalmic apparatuses such as a slit lamp microscope (a slit lamp), a corneal shape measurement apparatus, and others, for examining (including observing, measuring, and others) a patient's eye. For example, the slit lamp is arranged to project (irradiate) examination light formed of a slit shape (pattern) onto the eye, and allow an examiner to observe a section of a cornea sectioned by the slit examination light through a microscope. The corneal shape measurement apparatus is arranged to project (irradiate) examination light formed of a ring shape (pattern) having a plurality of concentric rings onto the eye, photographing (picking up) a ring image formed on a cornea, and analyzing it to measure the shape of the cornea.

The former slit lamp needs a structure to change a projection slit width and a projection slit direction of the slit examination light. Such structure, however, would make the apparatus complicated. The latter corneal shape measurement apparatus needs a structure to project the ring examination light; however, it would increase the apparatus in size.

### SUMMARY OF THE INVENTION

The present invention has been made in view of the above circumstances and to provide an ophthalmic apparatus capable of projecting examination light after forming the light into a desired projection shape (pattern) by a simple structure.

Additional objects and advantages of the invention will be set forth in part in the description which follows and in part will be obvious from the description, or may be learned by practice of the invention. The objects and advantages of the invention may be realized and attained by means of the instrumentalities and combinations particularly pointed out in the appended claims.

To achieve the purpose of the invention, there is provided an ophthalmic apparatus for examining an eye of a patient, comprising: a light projecting optical system for projecting examination light onto the eye, the optical system including: a light source; and a projection shape forming device which is electrically controlled to change a reflecting area of a reflecting surface or a transmitting area of a transmitting surface and is arranged to reflect or transmit the examination light emitted from the light source while forming a projection shape of the examination light into a desired shape so that the examination light of the desired shape is projected onto the eye; and a selection unit for selecting the projection shape of the examination light; and a control part which drivingly controls the forming device based on the selected projection shape.

### BRIEF DESCRIPTION OF THE DRAWINGS

The accompanying drawings, which are incorporated in and constitute a part of this specification illustrate an embodiment of the invention and, together with the description, serve to explain the objects, advantages and principles of the invention.

In the drawings,

FIG. 1 is a schematic perspective view of a slit lamp microscope in a first embodiment of the present invention;

FIG. 2 is a schematic structural view of an optical system and a control system of the slit lamp microscope;

FIGS. 3A to 3d are schematic views showing an example of drive control of micro mirrors of a micro-mirror device;

FIG. 4A is a schematic structural view of an optical system and a control system of a corneal shape measurement apparatus in a second embodiment of the invention;

FIG. 4B is a schematic view showing an example of drive control of micro mirrors of a micro-mirror device;

FIG. 5 is a schematic structural view of an optical system and a control system of a fundus shape measurement apparatus in a third embodiment of the invention; and

FIG. 6 is a schematic structural view of an optical system and a control system in a modified example of the corneal shape measurement apparatus.

### DETAILED DESCRIPTION OF THE PREFERRED EMBODIMENTS

A detailed description of a preferred embodiment of the present invention will now be given referring to the accompanying drawings.

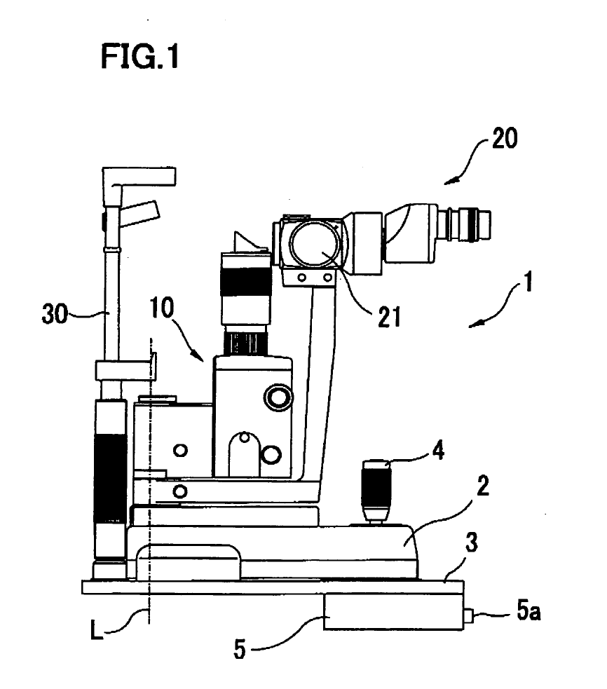

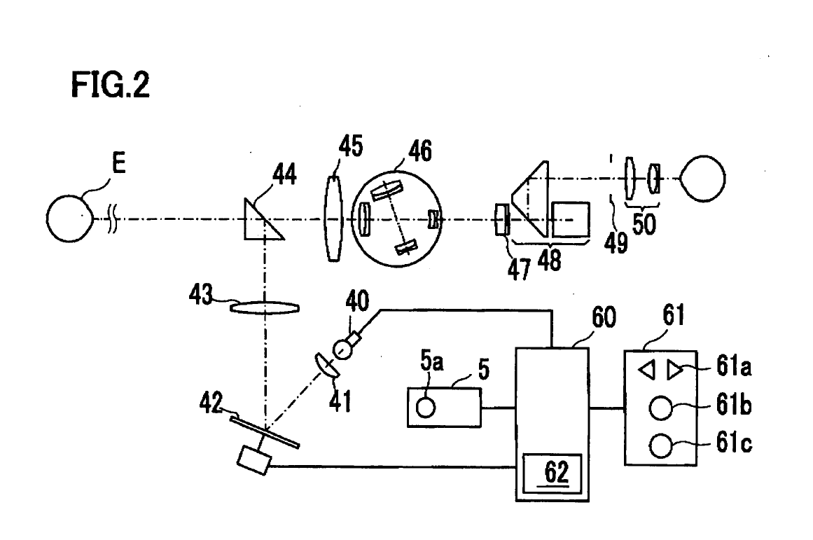

FIG. 1 is a schematic structural view of a slit lamp microscope (a slit lamp) in the first embodiment of the invention. FIG. 2 is a schematic structural view of an optical system and a control system of the slit lamp.

A slit lamp main unit 1 has a base 2 at a bottom thereof. This base 2 is mounted on a table 3 so that the base 2 is movable horizontally on the table 3 by a well known moving mechanism including a joystick 4. Secured to the table 3 is a head rest 30 provided with a chin piece, a forehead rest, and others, for fixedly supporting the face (the head) of an examinee (a patient) in position.

A control box 5 is provided with a power switch not shown, a light-adjusting knob 5a for adjusting a light amount of examination light, and others.

A projection part 10 for projecting (irradiating) the examination light onto an eye of the examinee includes therein a light projecting optical system mentioned later. This projection part 10 is rotatable about an axis L with respect to the base 2 so that the examinee may change a projection angle of the examination light as needed.

A microscope part 20 used to observe the examinee's eye includes therein an observation optical system mentioned later. A magnification changing knob 21 is turned to change an observation magnification to a desired one of 6-, 10-, 16-, 25-, and 40-power in the present embodiment. As with the projection part 10, the microscope part 20 is also rotatable about the axis L with respect to the base 2 so that the examinee may change an observation angle of the microscope part 20.

In the light projecting optical system, white visible examination (observation) light emitted from a light source 40 for examination (observation) passes through a condenser lens 41 and is reflected by a micro mirror device 42. This micro mirror device 42 is constructed of hundreds of thousand to few million of micro mirrors, each being movable independently, arranged on a CMOS semiconductor. Each micro mirror is deflected in a predetermined direction by control of voltage to be applied to each micro mirror. In the present embodiment, when the micro mirrors are deflected upon receiving the voltage, the examination light from the light source 40 is reflected toward the examinee's eye E and projected thereon. Specifically, the examination light reflected by the deflected micro mirrors of the micro mirror device 42 is reflected by a prism mirror 44 through a projection lens 43 toward the eye E.

The observation optical system includes the prism mirror 44 and an objective lens 45, both being used in common between a right and left observation optical paths, a variable magnification lens 46, an image forming lens 47, an erect prism 48, a field diaphragm 49, and eyepiece lenses 50, these components 46 to 50 being placed in each of the right and left observation optical paths.

Connected to a control part 60 which drivingly controls the whole main unit 1 are the light source 40, the micro mirror device 42, the control box 5, and a switch part 61 provided with various switches. The switch part 61 may be arranged separately from or attached (integrally) to the main unit 1. On the switch part 61, there are provided a projection slit width changing switch 61a for changing a projection slit width of the slit examination light to be formed by the micro mirror device 42, a projection slit direction changing dial 61b for changing a projection slit direction of the slit examination light, a projection slit pattern selection switch 61c for selecting a projection slit pattern (the number of slits) of the slit examination light, and others, In the present embodiment, the projection slit pattern of the slit examination light may be selected from among for example a single slit, a plurality of slits (three slits in the present embodiment), a cross-shaped slit, a full-area slit, but it is not limited thereto. A memory 62 stores in advance data for forming each projection shape by the micro mirror device 42 and other data.

Operation of the slit lamp having the above structure will be described below.

After prompting the examinee to put his head on the head rest 30 and the location of the eye E is identified, the examiner turns on the light source 40 to project the examination light to the eye E. To observe a section of the eye E, the examiner selects a desired projection slit pattern (the number of slits) with the switch 61c.

When the projection slit pattern (the number of slits) is selected with the switch 61c, the control part 60 reads the forming data corresponding to the selected projection slit pattern (the number of slits) from the memory 62 and, based on the data, drvingly control a deflecting motion of each micro mirror of the micro mirror device 42.

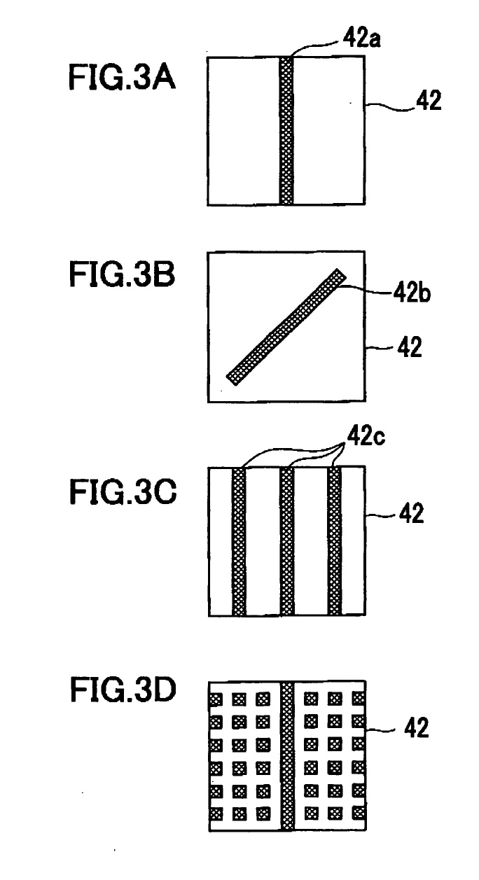

FIGS. 3A to 3D are schematic views each showing the driving control of each micro mirror of the micro mirror device 42 by the control part 60. A double hatched area in each figure represents micro mirrors deflected by applied voltage and an area not hatched represents another micro mirrors not deflected. In FIGS. 3A-3D, although the micro mirrors are not shown in detail, hundreds of thousands to few million of micro mirrors are practically arranged on a reflecting surface of the micro mirror device 42.

If the single slit is selected with the switch 61c, for instance, the control part 60 deflects only centrally arranged micro mirrors of the micro mirror device 42, i.e., micro mirrors 42a as shown in FIG. 3A.

By this deflection of parts of micro mirrors, the examination light reflected by the micro mirror device 42 toward the eye E is formed into a slit shape. The examiner turns the projection part 10 to appropriately adjust the projection angle so that the slit examination light formed (reflected) by the deflected micro mirrors of the micro mirror device 42 is obliquely projected onto the eye E. The examiner thus observes the section of the cornea of the eye E sectioned by the slit examination light, through the observation optical system.

To change the projection slit direction of the slit examination light, the examiner turns the dial 61b. The control part 60 deflects the micro mirrors of the micro mirror device 42 according to a turning angle of the dial 61b. For example, to incline the projection slit direction of the slit examination light by 45°, only the micro mirrors 42b which are parts of the micro mirrors of the micro mirror device 42 shown in FIG. 3D are deflected so that the slit examination light is brought into an 45°-inclined state without change in the projection slit width and the projection slit length of the slit examination light formed by the micro mirrors 42a shown in FIG. 3A. To change the projection alit width of the slit examination light, further, the examiner presses the switch 61a. The control part 60 then deflects the micro mirrors of the micro mirror device 42 to provide a projection slit width set with the switch 61a.

If wishing to observe an affected area extended in the cornea at a time, the examiner selects the plurality of slits with the switch 61c. When the plurality of slits is selected with the switch 61c, the control part 60 deflects only micro mirrors 42c of the micro mirror device 42 as shown in FIG. 3C.

If further wishing to observe an entire anterior segment of the eye E, the examiner selects the full-area slit with the switch 61c. When the full-area slit is selected by the switch 61c, the control part 60 deflects all the micro mirrors of the micro mirror device 42 to project the examination light onto substantially the entire area of the anterior segment of the eye E.

The use of the micro mirror device 42 also allows the following operations. For example, while deflecting part of the micro mirrors of the micro mirror device 42 to form the slit examination light into a single slit, the control part 60 selectively deflects the other micro mirrors not used to form the slit examination light at very short time intervals (e.g., 1 ms), as shown in FIG. 3D. This makes it possible to simultaneously project the slit examination light and background illumination light which is lower in light amount than the slit examination light. In addition, the control part 60 can deflect the micro mirrors of the micro mirror device 42 to project the slit examination light while making it scan (for example, so as to scan from left to right and/or from above to below). Such driving control of the micro mirror device 42 by the control part 60 can be set with the switch 61c.

In the slit lamp in the present embodiment, as above, the micro mirrors of the micro mirror device 42 are deflected to provide a desired projection shape of the examination light according to observation purposes. Accordingly, various observing manners can be presented by a simple structure as compared with the conventional slit lamp.

In the present embodiment, the micro mirror device is used to form the projection shape of the slit examination light, but it is not limited thereto. Any other device, e.g., spatial light modulator (SLM) devices, capable of forming the projection shape of light by control of a light reflecting or transmitting area may be used. Although the white examination light is projected in the present embodiment, instead thereof, color (e.g., blue) examination light for fluorescent observation may be projected. In this case, a filter of three colors (red, green, and blue) is disposed to be rotatable between the light source 40 and the micro mirror device 42. If the white examination light is required, the filter is rotated at high speed to produce white light by combining light having passed through the three-color filter. If the color examination light is required, on the other hand, the micro mirrors of the micro mirror device 42 are deflected in time with (in sync with) the time when light passes through a desired color of the three-color filter so that only the desired color light is reflected toward the eye E.

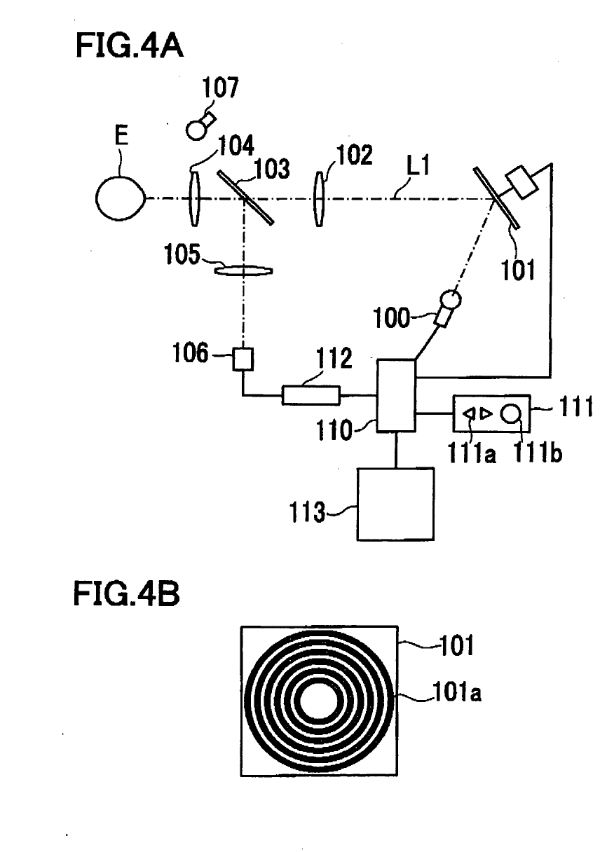

FIG. 4A is a schematic structural view of an optical system and a control system of a corneal shape measurement apparatus in a second embodiment of the present invention.

The optical system for measuring a corneal shape of the examinee's eye E comprises a light projecting optical system including a light source 100 for examination (measurement), a micro mirror device 101, a lens 102, a beam splitter (a half mirror) 103, and a lens 104; and a light receiving optical system including the lens 104, the beam splitter 103, a lens 105, and a CCD camera 106 provided with an image pickup device which has the sensitivity to wavelengths in a visible to infrared range. Examination light emitted from the light source 100 is reflected along an optical axis L1 by deflected micro mirrors of the micro mirror device 101. The examination light reflected along the optical axis L1 passes through the lens 102, the beam splitter 103, and the lens 104 and then is projected onto the eye E. This apparatus is arranged to project the examination light in a ring pattern having a plurality of concentric rings or in a grid pattern (a mesh pattern) onto the eye E. The lenses 102 and 104 are each designed to have an appropriate lens diameter, refractive power, set position, and others so that the examination light reflected by the deflected micro mirrors of the micro mirror device 101 is directed to the cornea of the eye E and also the examination light reflected by the cornea is directed to the lens 105 through the lens 104 and the beam splitter 103. A ring or grid image formed on the cornea is picked up (photographed) by the camera 106 through the lens 104, the beam splitter 103, and the lens 105. Numeral 107 denotes an infrared light source for illuminating the anterior segment of the eye E.

Connected to a control part 110 are the light source 100, the micro mirror device 101, a switch part 111 provided with various switches, an image processing part 112, a display 113, and others. The switch part 111 is provided with a selection switch 111a for selection of a projection shape (pattern) of the examination light, a measurement start switch 111b, and others. In the present embodiment, the projection shape (pattern) of the examination light can be selected between two types; the ring pattern and the grid pattern.

Operation of the corneal shape measurement apparatus having the above structure will be described below.

The examiner selects the projection shape (pattern) of the examination light with the switch 111a. In the following description, assume that the ring pattern is selected. After selection of the projection shape, the examiner turns on the light source 107 so that an image of the anterior segment of the eye E is displayed on the display 113, and makes alignment of the apparatus with respect to the eye E by use of a well known alignment system not shown. After completion of alignment, the examiner presses the switch 111b to start measurement.

Upon pressure of the switch 111b, the control part 110 turns on the light source 100 and deflects micro mirrors 101a which are parts of the micro mirrors of the micro mirror device 101 as shown in FIG. 4B, thereby projecting the examination light in a ring pattern having a plurality of concentric rings At this time, the light from the light source 100 is projected onto the entire reflecting surface of the micro mirror device 101. The examination light reflected by the micro mirrors 101a passes through the lens 102, the beam splitter 103, the lens 104 and is projected onto the cornea, forming thereon a plurality of ring images. The ring images are picked up (photographed) by the camera 106 through the lens 104, the beam splitter 103, and the lens 105.

An image signal outputted from the camera 106 is stored in a frame memory not shown and then outputted to the image processing part 112. The image processing part 112 performs edge detection on the picked-up ring images to acquire each edge position with respect to a corneal center in steps of a predetermined angle (e.g., 1°), thereby analyzing a distribution of corneal curvature. After the analysis in the image processing part 112, the control part 110 stores a measurement result in a memory not shown and displays the acquired corneal shape in the form of a map on the display 113. This corneal shape data can be used as for example data for corneal correction with a cornea correcting surgical apparatus using an excimer laser beam.

In the above embodiment, the projection shape (pattern) of the examination light is selected between the ring pattern and the grid pattern. Instead of such pattern, a two-dimensional pattern for measuring a corneal shape may be used. Further, the width of each projection ring, the number of projection rings, the interval of projection rings, and/or the width of a projection grid (a square of a grid pattern), the interval of projection grids (squares), and others may be changed as appropriate.

It is further possible to select, with time, the micro mirrors to be deflected in one measurement, thereby changing the diameter of the ring examination light to be projected onto the eye E while sequentially picking up (photographing) the ring images formed on the cornea by the camera 106, and accumulating analysis data obtained from each ring image to acquire the shape of the entire cornea. In the case where the examination light is projected in a ring pattern having many rings as in the conventional apparatus, the ring images formed on the cornea may largely be deformed depending on the corneal shape. This would cause the ring images to partially overlap one another, which makes it difficult to accurately perform the edge detection of the ring images. Thus, an analysis result with high precision may not be obtained. To avoid such disadvantage, it is preferable to use a single ring or the low number of rings (the number of rings that will not cause overlapping of the ring images) as the ring image to be picked up by the camera at a time and change with time the diameter of the ring image(s) to be formed on the cornea so that the number of ring images to be picked up by the camera within a predetermined time is increased. By using this manner, analysis errors resulting from the overlapping of the ring images can be prevented and therefore the corneal shape can be measured in detail.

Although the micro mirror device is used in the present embodiment, it is not limited thereto, As in the first embodiment mentioned above, any other device, e.g., spatial light modulator (SLM) devices, capable of forming the projection shape of light by control of a light reflecting or transmitting area may be used.

In the second embodiment, the apparatus is arranged to project the examination light onto the cornea to measure the corneal shape, but it is not limited thereto. It may be arranged to project the examination light to a fundus of the eye E to measure a shape of the fundus (a retina).

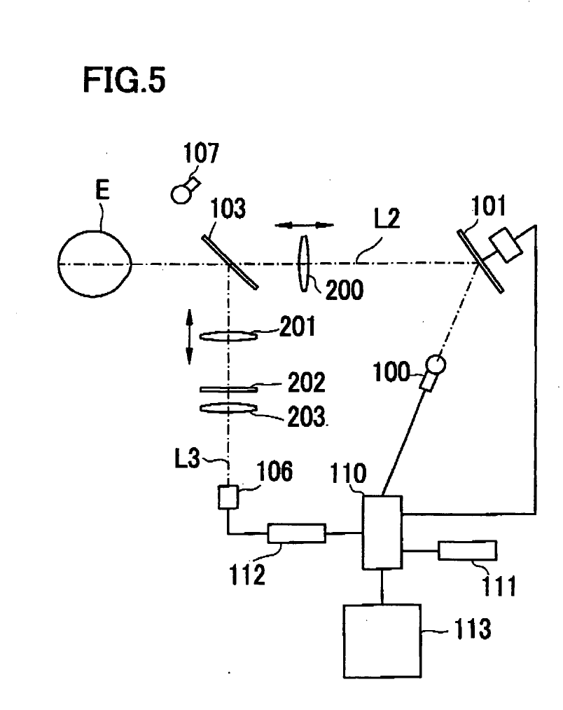

FIG. 5 is a schematic structural view of an optical system and a control system of a fundus shape measurement apparatus in a third embodiment of the present invention. In FIG. 5, components or elements having the same functions as those in FIG. 4A are indicated by the same numerals and their explanations are omitted here.

A lens 200 is placed between the micro mirror device 101 and the beam splitter 103 to direct the examination light reflected along an optical axis L2 by the deflected micro mirrors the micro mirror device 101 toward the fundus of the eye E, forming an image of the examination light on the fundus. The lens 200 is movable along the optical axis L2 by a lens moving mechanism not shown for adjustment to a refractive power of the eye E.

Reflection light from the fundus is reflected by the beam splitter 103 and forms a spatial image at an image conjugate position through the lens 201. The light forming the spatial image passes through a black spot plate 202 having a small black spot at a center and then is picked up (photographed) by the camera 106 through the lens 203. The black spot plate 202 is disposed in a substantially conjugate relationship with respect to a corneal reflection luminescent spot of the eye E through the lens 201, thereby interrupting the reflection light from the corneal surface, preventing it from entering as noise light into the camera 106. The lens 201 is arranged to be movable along an optical axis L3 in conjunction with the lens 200.

With the above structure, the examiner selects the projection shape (pattern) of the examination light with the switch part 111, makes alignment, and presses the measurement start switch. Upon pressure of the measurement start switch, the control part 110 turns on the light source 100 and deflects parts of the micro mirrors of the micro mirror device 101 to project the examination light of the selected projection shape. The light from the light source 100 is reflected by the deflected micro mirrors of the micro mirror device 101 and is projected onto the fundus through the lens 200 and the beam splitter 103, forming an image on the fundus. The image formed on the fundus is picked up by the camera 106 through the beam splitter 103, the lens 201, the black spot plate 202, and the lens 203. The image processing part 112 analyzes the shape of the fundus (the retina) based on the image picked up by the camera 106 and displays the acquired fundus shape in the form of a map on the display 113.

In the first through third embodiments described above, it is preferable to use a spatial light modulator device such as a liquid-crystal spatial light modulator and a deformable mirror device, instead of using the micro mirror device. The spatial light modulator device can control the light reflecting or transmitting area and correct the wavefront of light (distortion of light) as well. When irregularities of the wavefront of the examination light emitted from the light source are corrected, the projection light can be formed into a high contrast projection shape with sharp edges. This makes it possible to enhance the precision of observation and measurement. If the wavefront of the examination light has to be corrected, a wavefront sensor is provided to detect the wavefront of the examination light.

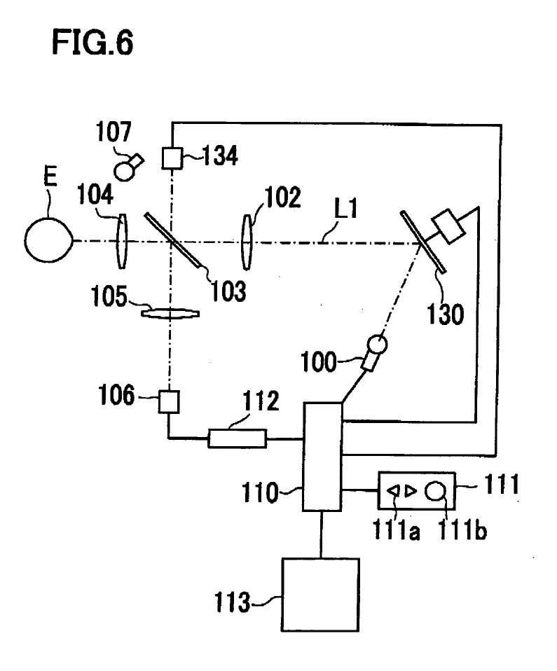

FIG. 6 is a modified example of the corneal shape measurement apparatus shown in FIG. 4A. In this example, instead of the micro mirror device, a reflecting type spatial light modulator (SLM) device 130 is used. A wavefront sensor 134 is disposed on the optical path along which the examination light is reflected by the beam splitter 103. The control part 110 drivingly controls the SML device 130 based on a detection result of the wavefront sensor 134 to adjust the wavefront of tho examination light of the projection shape (a ring pattern, a grid pattern, etc.). The control for correcting the wavefront by using the SLM device 130 and the wavefront sensor 134 can also apply to the optical system of the slit lamp shown in FIG. 2 and the optical system of the fundus shape measurement apparatus shown in FIG. 5.

In the present embodiments as mentioned above, the slit lamp for observing the examinee's eye, the corneal shape measurement apparatus, and the fundus shape measurement apparatus are explained respectively. Even a single ophthalmic apparatus having all the observation function, corneal shape measurement function, and fundus shape measurement function can be achieved by using, as a common component, a device capable of controlling the light reflection or transmission to project examination light of a desired shape onto the examinee's eye, such as the micro mirror device and the spatial light modulator device, and incorporating an optical system for observing the examinee's eye and measuring the corneal shape and fundus shape.

While the presently preferred embodiment of the present invention has been shown and described, it is to be understood that this disclosure is for the purpose of illustration and that various changes and modifications may be made without departing from the scope of the invention as set forth in the appended claims.

1. An ophthalmic apparatus for examining an eye or a patient, comprising:
a light projecting optical system for projecting examination light onto the eye, the optical system including:
a light source; and
a projection shape forming device which is electrically controlled to change a reflecting area of a reflecting surface or a transmitting area of a transmitting surface and is arranged to reflect or transmit the examination light emitted from the light source while forming a projection shape of the examination light into a desired shape so that the examination light of the desired shape is projected onto the eye; and
a selection unit for selecting the projection shape of the examination light; and
a control part which drivingly controls the forming device based on the selected projection shape.

2. The ophthalmic apparatus according to claim 1, wherein
the light projecting optical system can project the examination light of a slit shape onto the eye, and
the selection unit selects at least one of a slit width, a slit direction, and the number of slits of the slit examination light to be projected.

3. The ophthalmic apparatus according to claim 1, wherein
the light projecting optical system can project the examination light of a ring shape onto the eye, and
the selection unit selects at least one of a ring width, the number of rings, and an interval of rings of the ring examination light to be projected.

4. The ophthalmic apparatus according to claim 3 farther comprising:
a light receiving optical system which includes an image pickup device and receives reflection light of the projected ring examination light from an examined portion of the eye; and
an analysis part which processes an image picked up by the image pickup device to acquire a shape of the examined portion.

5. The ophthalmic apparatus according to claim 1, wherein
the light projecting optical system can project the examination light of a grid shape onto the eye, and
the selection unit selects at least one of a grid width and a grid interval or the grid examination light to be projected.

6. The ophthalmic apparatus according to claim 5 further comprising:
a light receiving optical system which includes an image pickup device and receives reflection light of the projected grid examination light from an examined portion of the eye; and
an analysis part which processes an image picked up by the image pickup device to acquire a shape of the examined portion.

7. The ophthalmic apparatus according to claim 1, wherein the forming device includes a micro mirror device having micro mirrors whose deflecting directions can be changed individually.

8. The ophthalmic apparatus according to claim 1, wherein the forming device includes a spatial light modulator device which corrects a wavefront of light.

9. The ophthalmic apparatus according to claim 8 further comprising a wavefront sensor which detects the wavefront of the examination light to be projected,
wherein the control part drivingly controls the spatial light modulator device based on a detection result of the wavefront sensor.
